(19) United States
(12) Patent Application Publication
(10) Pub. No.: US 2026/0215672 A1
(43) Pub. Date: Jul. 30, 2026

(54) ORAL IMAGING DEVICE AND SYSTEM THEREOF

(71) Applicant: GUANGZHOU STARS PULSE CO., LTD., Guangzhou (CN)

(72) Inventors: Hang FAN, Guangzhou (CN); Lulu NING, Guangzhou (CN); Hanhong DAI, Guangzhou (CN); Hongquan MO, Guangzhou (CN); Bo CHEN, Guangzhou (CN)

(73) Assignee: GUANGZHOU STARS PULSE CO., LTD., Guangzhou (CN)

(21) Appl. No.: 19/209,749

(22) Filed: May 15, 2025

Related U.S. Application Data
(63) Continuation of application No. PCT/CN2025/075430, filed on Jan. 27, 2025.

(30) Foreign Application Priority Data
Apr. 30, 2025 (CN) 202510571048.6

(51) Int. Cl. A61B 1/247; A61B 1/00; A61B 1/05; A61B 1/06; A61B 1/32

(52) U.S. Cl. CPC A61B 1/247; A61B 1/00154; A61B 1/00174; A61B 1/05; A61B 1/0623; A61B 1/32

(57) ABSTRACT

An oral imaging device and an oral imaging system are provided. The oral imaging device includes a body, a support arm coupled to the body, and a camera module positioned at an intra-oral end of the support arm and configured to capture at least a first image including at least a first portion of a first dental arch. The support arm includes the intra-oral end and a first guide structure. And the support arm is stabilized by the first guide structure when the oral imaging device rests on a second dental arch.

### CROSS-REFERENCE TO RELATED APPLICATION

This application claims the benefit of priority to Chinese Patent Application No. 202510571048.6, filed on Apr. 30, 2025. This application is also a continuation of International Application No. PCT/CN 2025/075430, filed on Jan. 27, 2025. The entire contents of these applications are hereby incorporated by reference in their entireties.

### BACKGROUND

The present disclosure relates to an oral imaging device and a system thereof, and in particular to a handheld, electrically powered oral imaging device and a system controlling the oral imaging device.

An oral imaging device can be utilized by a user or dentist to capture images of a patient's mouth or teeth for further examination or diagnosis. Conventional oral imaging devices typically capture images of one or a few teeth at a time and are unable to capture the entire dental arch in a single image. Additionally, some oral imaging devices are limited to capturing the external surfaces of the teeth or dental arch, making them incapable of imaging the inner surfaces, which restricts their overall functionality.

To capture the entire dental arch in a single image, a dentist might opt for a professional Single Lens Reflex (SLR) camera and a reflex mirror for photography, a process that involves more complex equipment and procedures. A simpler and more user-friendly camera device capable of scanning the entire dental arch is needed.

### SUMMARY

In one aspect, an oral imaging device includes a body; a support arm coupled to the body; and a camera module positioned at an intra-oral end of the support arm and configured to capture at least a first image including at least a first portion of a first dental arch. The support arm includes the intra-oral end and a first guide structure. And the support arm is stabilized by the first guide structure when the oral imaging device rests on a second dental arch.

In some implementations, the camera module is configured to move along a first trajectory.

In some implementations, the support arm further includes a second guide structure. The camera module is configured to capture at least a second image including at least a second portion of the first dental arch or at least a portion of the second dental arch, and the support arm is stabilized by the second guide structure when the oral imaging device rests on the first dental arch or the second dental arch.

In some implementations, the camera module is configured to move along a second trajectory, and a plane of the second trajectory being different from that of the first trajectory.

In some implementations, the plane of the second trajectory and the plane of the first trajectory are perpendicular to each other.

In some implementations, the support arm further includes a third guide structure. The support arm is stabilized by the third guide structure when the oral imaging device rests on the same side as the first dental arch or the second dental arch, in order to capture at least a portion of the first dental arch or a portion of the second dental arch.

In some implementations, the third guide structure is on a first surface facing a first direction to which an aperture of a camera of the camera module is facing.

In some implementations, the oral imaging device is configured to rest on an outer side of teeth or a jaw via the third guide structure to shoot the outer side of the first dental arch or the second dental arch.

In some implementations, the third guide structure is configured to lift the camera module away from an outer surface of the first dental arch or the second dental arch so that there is a threshold distance between the camera module and the outer surface of the first dental arch or the second dental arch for capturing images.

In some implementations, the third guide structure is closer to the first dental arch than the camera module when the camera module rests on the first dental arch.

In some implementations, the third guide structure includes at least one of a convex or a bending structure.

In some implementations, the third guide structure includes at least one rounded surface in contact with the first dental arch.

In some implementations, the first guide structure is on a second surface facing a second direction opposite to a first direction to which the aperture of the camera of the camera module is facing.

In some implementations, the oral imaging device is configured to rest on an anterior tooth of the first dental arch via the first guide structure to capture an occlusal surface of the second dental arch.

In some implementations, the first guide structure is configured to control the depth at which the camera module is inserted into the oral cavity or maintain a distance between the camera and the first dental arch to be captured.

In some implementations, the first guide structure includes at least one of a concave, a convex, or a bending structure.

In some implementations, the concave includes at least one of a plurality of dot holes or a plurality of trenches.

In some implementations, the convex includes at least one of a plurality of dot protrusions, or a plurality of strip protrusions.

In some implementations, a depth of the concave is equal to or less than 3.5 mm.

In some implementations, a width of the concave is equal to or less than 8 mm.

In some implementations, the support arm has a first surface, a second surface, and a third surface, the second surface is opposite to the first surface, and the third surface connects the first surface and the second surface, and the camera module is on the first surface, and the second guide structure is on the third surface.

In some implementations, the third surface and the first surface are perpendicular to each other, and the first surface and the second surface are parallel to each other.

In some implementations, the third surface is smooth except for the second guide structure.

In some implementations, a thickness of the camera module is equal to or less than 12 mm.

In some implementations, the second guide structure includes a plurality of concaves, or a plurality of convexes, or a combination thereof, configured to accommodate dental arches of different sizes.

In some implementations, the support arm is pillar shaped, and a distance between the camera module and the body is at least 20 mm.

In some implementations, a surface of the first guide structure contacting the dental arch is smooth in a width direction.

In some implementations, the camera module includes: a camera including a wide-angle camera.

In some implementations, the camera module includes a plurality of lightings spaced apart and surrounding the camera.

In some implementations, the camera module includes: a lens hood surrounding the camera and positioned between the camera and the plurality of lightings.

In some implementations, a field of view (FOV) of the camera is equal to or larger than 140°.

In some implementations, the camera has a rectangular FOV, and a shorter side of the rectangular FOV is parallel to a length direction of the support arm.

In some implementations, an angle between an optical axis of the camera and an occlusal surface of a patient's oral cavity is between 60° to 90° when the oral imaging device rests on the second dental arch.

In some implementations, an angle between the optical axis of the camera and a length direction of the body is between 40° to 90°.

In some implementations, the optical axis of the camera is tiled away from the body.

In some implementations, the plurality of lightings is configured to generate a light including at least one of a white light, a blue light, a purple light, an ultraviolet (UV) light, or a red light.

In some implementations, the plurality of lightings is configured to generate a light including a wavelength in a range selected from at least one of the following ranges: 315 nm to 400 nm, 380 nm to 750 nm, 450 nm to 500 nm, or 750 nm to 1,500 nm.

In some implementations, the camera and the plurality of lightings are arranged so that a distance between the camera and an intersection of light paths of the plurality of lightings is equal to or less than 10 mm. The camera and the plurality of lightings are arranged so that a distance between the position where the light paths of the plurality of lightings and the FOV of the camera are offset to the camera is equal to or greater than 30 mm.

In some implementations, the oral imaging device further includes: a controller coupled to the body. The controller is configured to operate the camera and the plurality of lightings.

In some implementations, the oral imaging device further includes: a power source inside the body. The power source provides electric power to the controller within the body.

In some implementations, the oral imaging device further includes: a power source provided separately from the body and providing electric power to the body via a wired or wireless connection.

In some implementations, the oral imaging device further includes: a memory coupled to the body. The memory is configured to store the images captured by the camera.

In some implementations, the camera module includes: at least two cameras; and at least two groups of lightings, each group correspondingly surrounding one of the at least two cameras.

In some implementations, an intersection angle between the optical axes of two of the at least two cameras is between 130° and 160°.

In some implementations, the oral imaging device further includes: a gag, and an opening of the gag is configured to correspond to an aperture of a camera of the camera module to allow the camera to capture the image via the opening of the gag.

In some implementations, a relative position between the gag and the support arm is adjustable.

In some implementations, the gag is coupled to the body.

In some implementations, the oral imaging device further includes: a housing coupled to the body, and the gag is coupled to the housing or is a part of the housing.

In some implementations, the gag includes a main portion and an extension portion, and the extension portion is disposed on one end of the gag away from the housing.

In some implementations, the housing is detachably coupled to the body on a first surface of the body, and the support arm extends away from the first surface of the body.

In some implementations, the housing is detachably coupled to the body on a second surface of the body, and the second surface of the body and the aperture of the camera face the same direction.

In some implementations, the housing is configured to slide away from or close to the body in a first direction, and the support arm extends away from the body in the first direction.

In some implementations, the housing is sized to accommodate the support arm.

In some implementations, the support arm is rotatable via a shaft coupled to the body.

In some implementations, the support arm is configured to be accommodated in a groove of the body when the support arm is folded, and the support arm is configured to extend away from the body when the support arm is unfolded.

In some implementations, the support arm is detachably coupled to the body.

In another aspect, an oral imaging device includes: a body; a support arm coupled to the body and a camera module positioned at an intra-oral end of the support arm. The camera module includes: a camera, and an FOV of the camera is equal to or larger than 140°.

In some implementations, the camera module further includes: a plurality of lightings spaced apart and surrounding the camera; and a lens hood surrounding the camera and positioned between the camera and the plurality of lightings.

In some implementations, the camera has a rectangular FOV, and a shorter side of the rectangular FOV is parallel to a length direction of the support arm.

In some implementations, an angle between an optical axis of the camera and an occlusal surface of a patient's oral cavity is between 60° to 90° when the oral imaging device rests on a dental arch.

In some implementations, an angle between the optical axis of the camera and a length direction of the body is between 40° to 90°.

In yet another aspect, an oral imaging device includes: a body; a support arm coupled to the body, and a camera module positioned at an intra-oral end of the support arm and configured to capture at least a first image including at least a portion of a first dental arch of an oral cavity. A position of the camera module in the oral cavity is limited by a position of a first guide structure of the support arm placed on a second dental arch of the oral cavity.

In some implementations, the camera module is positioned away from the first dental arch by placing the first guide structure on the second dental arch.

In some implementations, the support arm further includes a second guide structure. The camera module is configured to capture at least a second image including at least a portion of the first dental arch or at least a portion of the second dental arch. The position of the camera module in the oral cavity is limited by a position of the second guide structure placed on the second dental arch.

In some implementations, the support arm further includes a third guide structure on a first surface of the support arm. The first guide structure is on a second surface of the support arm facing opposite to the first surface. A depth of the camera module relative to the second surface is less than a depth of the third guide structure relative to the second surface.

### BRIEF DESCRIPTION OF THE DRAWINGS

The accompanying drawings, which are incorporated herein and form a part of the specification, illustrate aspects of the present disclosure and, together with the description, further serve to explain the principles of the present disclosure and to enable a person skilled in the pertinent art to make and use the present disclosure.

FIGS. 1A-1H illustrate block diagrams of an exemplary oral imaging device and its corresponding operating states, according to some implementations of the present disclosure.

FIGS. 2A-2C illustrate exemplary scanning shots for oral scanning operations, according to some implementations of the present disclosure.

FIGS. 3A-3N illustrate block diagrams of exemplary oral imaging devices and their corresponding operating states, according to some implementations of the present disclosure.

FIGS. 4A-4B illustrate block diagrams of an exemplary oral imaging device and its corresponding operating states, according to some implementations of the present disclosure.

FIGS. 5A-5B illustrate block diagrams of a camera module of an exemplary oral imaging device and its corresponding light path during the operations, according to some implementations of the present disclosure.

FIG. 6 illustrates a block diagram of an exemplary oral imaging system, according to some implementations of the present disclosure.

The present disclosure will be described with reference to the accompanying drawings.

### DETAILED DESCRIPTION

Although specific configurations and arrangements are discussed, it should be understood that this is done for illustrative purposes only. A person skilled in the pertinent art will recognize that other configurations and arrangements can be used without departing from the spirit and scope of the present disclosure. It will be apparent to a person skilled in the pertinent art that the present disclosure can also be employed in a variety of other applications.

It is noted that references in the specification to “one embodiment,” “an embodiment,” “an example embodiment,” “some implementations,” etc., indicate that the embodiment described may include a particular feature, structure, or characteristic, but every embodiment may not necessarily include the particular feature, structure, or characteristic. Moreover, such phrases do not necessarily refer to the same embodiment. Further, when a particular feature, structure or characteristic is described in connection with an embodiment, it would be within the knowledge of a person skilled in the pertinent art to affect such feature, structure or characteristic in connection with other implementations whether or not explicitly described.

In general, terminology may be understood at least in part from usage in context. For example, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

It should be readily understood that the meaning of “on,” “above,” and “over” in the present disclosure should be interpreted in the broadest manner such that “on” not only means “directly on” something but also includes the meaning of “on” something with an intermediate feature, a layer, or a structure therebetween, and that “above” or “over” not only means the meaning of “above” or “over” something but can also include the meaning it is “above” or “over” something with no intermediate feature, layer, or structure therebetween (i.e., directly on something).

In addition, range-related terms, such as “between A and B,” “a range between A and B,” “from A to B,” and the like, are used herein for convenience to describe “a range from A to B, inclusive,” indicating that both A and B are included within the range, unless A and/or B are specifically excluded.

Further, spatially relative terms, such as “beneath,” “below,” “lower,” “above,” “upper,” and the like, may be used herein for ease of description to describe one element or a feature's relationship to another element(s) or feature(s) as illustrated in the figures. The spatially relative terms are intended to encompass different orientations of the device in use or operation in addition to the orientation depicted in the figures. The device may be otherwise oriented (rotated 90 degrees or at other orientations) and the spatially relative descriptors used herein may likewise be interpreted accordingly. In addition, the term “couple”, “coupled to”, or “coupled between” may be understood as not necessarily intended to be “physically joined or attached,” i.e., direct attachment, but can also be interpreted as indirect connection through an intermediate component.

As mentioned above, an oral imaging device can be utilized by a user or dentist to capture images of a patient's oral cavity or teeth for further examination or diagnosis. Conventional oral imaging devices typically capture images of one or a few teeth at a time and are unable to capture the entire dental arch in a single image. Additionally, some oral imaging devices are limited to capturing the external surfaces of the teeth or dental arch, making them incapable of imaging the inner surfaces, which restricts their overall functionality. Generally, oral imaging devices can be categorized into two types. The first type is a rod-shaped entry-type oral imaging device, designed to capture images of one or a few teeth at a time, but it cannot scan and capture the entire dental arch in a single image. The second type is a mouth opener-type oral imaging device, which can take images of the outer surfaces of the dental arch but is unable to capture the entire inner surfaces with clear view, leading to limited usage.

To address one or more of the aforementioned issues, this disclosure presents handheld and adjustable entry-type oral imaging devices that enable the user or dentist to adapt the devices to different scenarios and capture images of the inner and outer surfaces of the dental arch. It is a compact, portable, and lightweight oral imaging device that can be easily held and operated by hand without the need for additional mechanical assistance. No extra mirror is required to capture images of the interior surfaces of the dental arch. In some implementations, the oral imaging device can include various types of guide structures that are used to limit the position of the camera within the patient's oral cavity and/or stabilize the camera. It is noted that the oral imaging device according to the present disclosure can be operated by the user to examine his or her own oral cavity, and thus, in such instances, the patient and the user are the same individual. In some implementations, these guide structures allow the camera to be inserted into the lower part of the patient's oral cavity to capture images of the upper dental arch, or into the upper part to capture images of the lower dental arch. With help of the present disclosure, each tooth surface would have better direct shooting angles, rather than limited angles in conventional oral imaging devices. Additionally, these guide structures can restrict the camera's insertion positions, providing better positions and angles for capturing the occlusal surfaces of the teeth, and offering different options for both adult and child patients. For example, the relative positions of these guide structures and the camera are tailored to accommodate varying dental arch sizes or oral cavity dimensions, such as those of adults and children or individuals with larger or smaller builds. For instance, it can be designed into S, M, and L sizes.

Moreover, these guide structures enable users or dentists to move the oral imaging device along a preset trajectory while it is stabilized on the dental arch, allowing image capture tailored to different examination scenarios. The users or dentist may use the guide structure disclosed herein as a fulcrum for a seesaw-like rotation to move along the trajectory, and thus the camera's shooting angle may be adjusted along the trajectory, allowing it to capture images at a specific angle or position relative to the opposing occlusal surface. For example, these guide structures allow the users or dentists to take pictures of inner surfaces of dental arch, outer surfaces of dental arch, upper dental arch, lower dental arch, a portion of the upper dental arch, a portion of the lower dental arch, one or a few teeth from inside or outside of the oral cavity, etc. It is noted that in addition to capturing images of the teeth or dental arches, the present disclosure can also capture images of at least part of the periodontal tissue, tongue, or oral mucosa.

In some implementations, the support arm features various designs to enhance flexibility and accessibility for the user, thereby improving the overall user experience. Specifically, it may simplify the user's process of operations, save shooting time, enhance patient's comfort, and allow users to operate the oral imaging device themselves to capture images of their own oral condition. In some implementations, a gag design is incorporated into the oral imaging device to improve the flexibility and ease of installment, replacement, or detachment. Furthermore, when capturing an image with a flash (e.g., in the form of one or more lightings, including fill lights, functional lights, etc.), the camera lens or a surrounding lens hood may block the light from the flash, resulting in dark shadows around the object. Therefore, in some implementations, the camera and its lightings are designed to enhance illumination and minimize dark shadows, spots, or edges. In addition, the lightings can be used for enhancing the visualization of pathological conditions by providing sufficient exposure dosage, resulting in clear images of targeted oral areas.

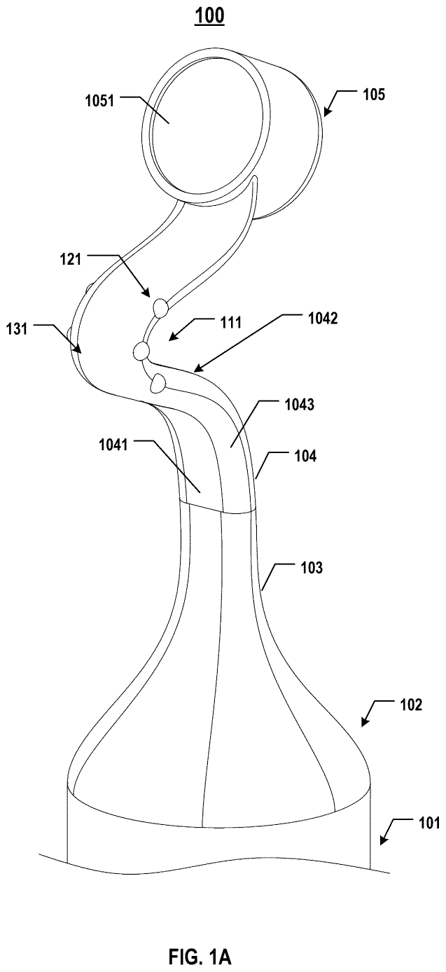

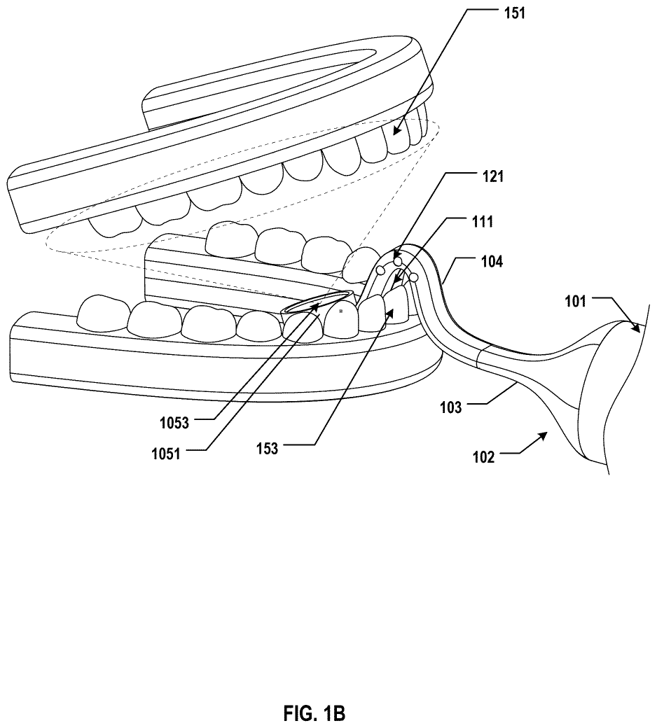

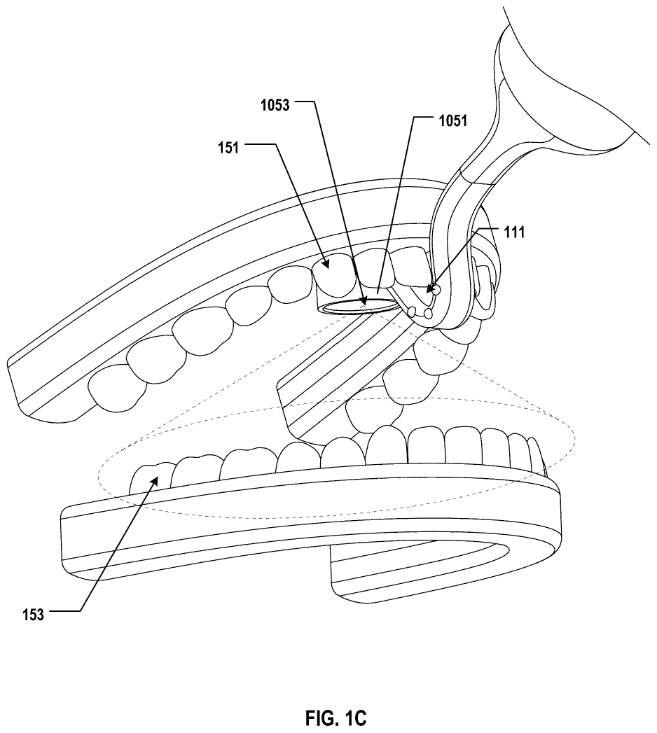

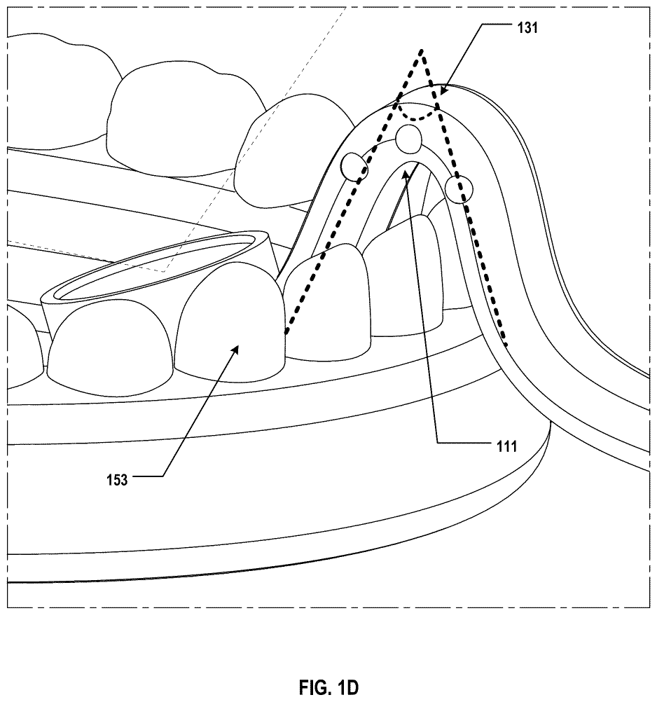

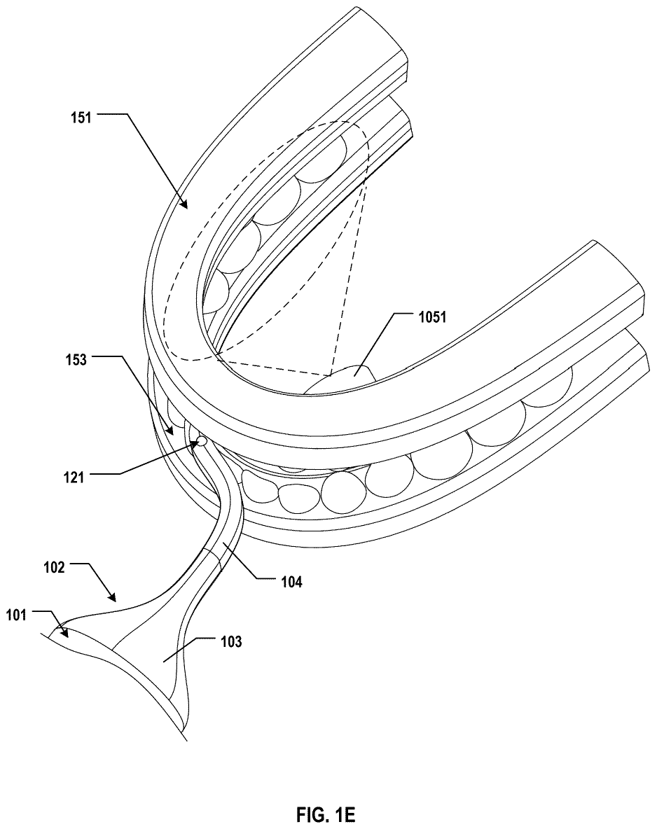

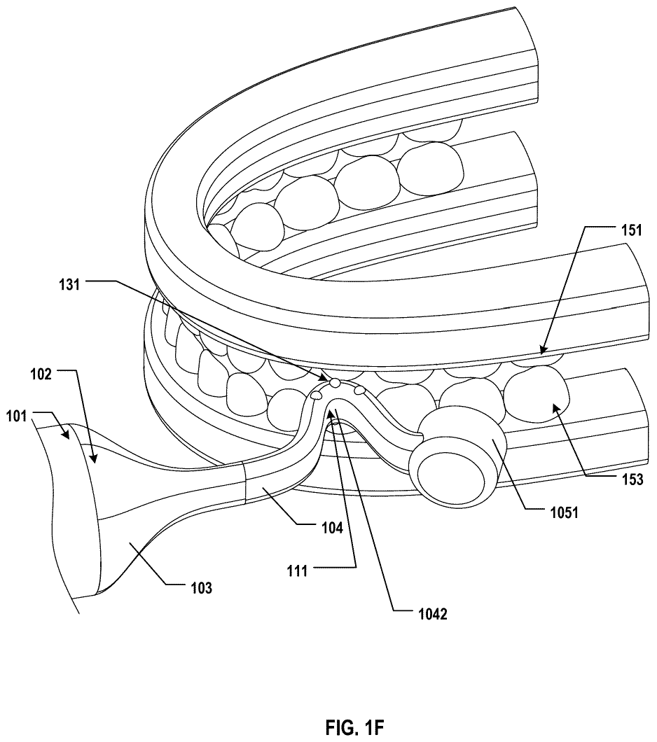

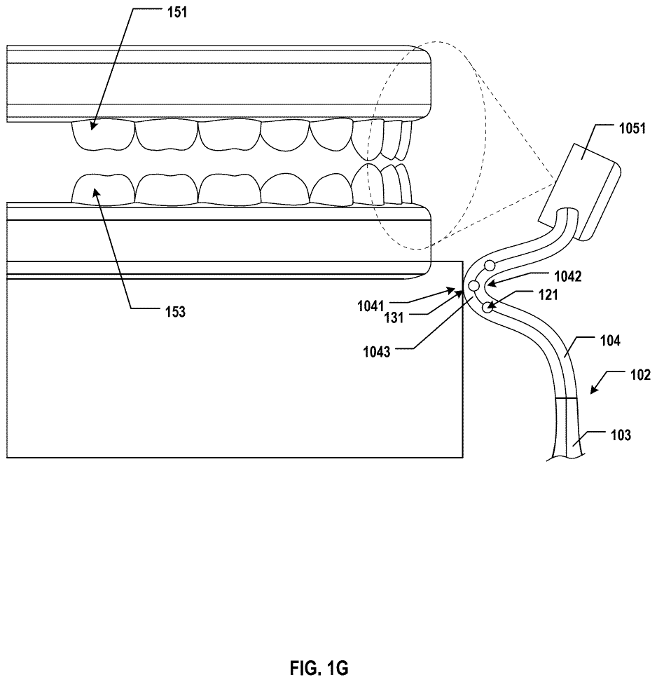

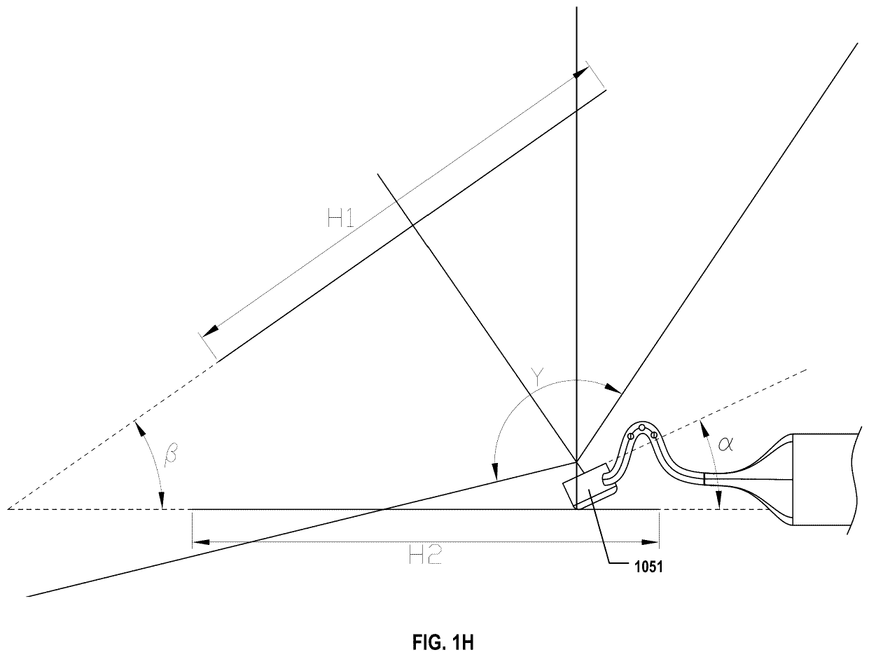

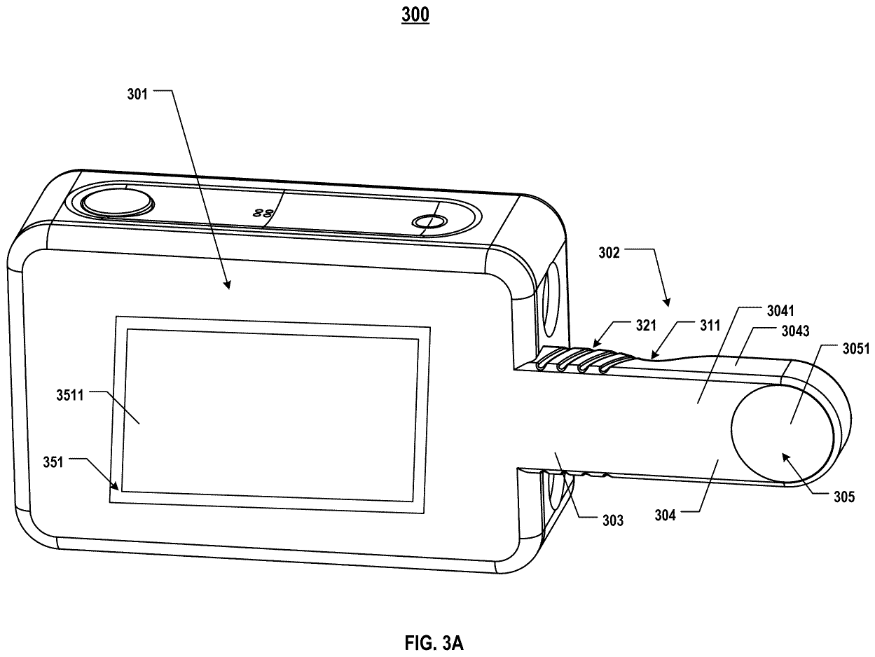

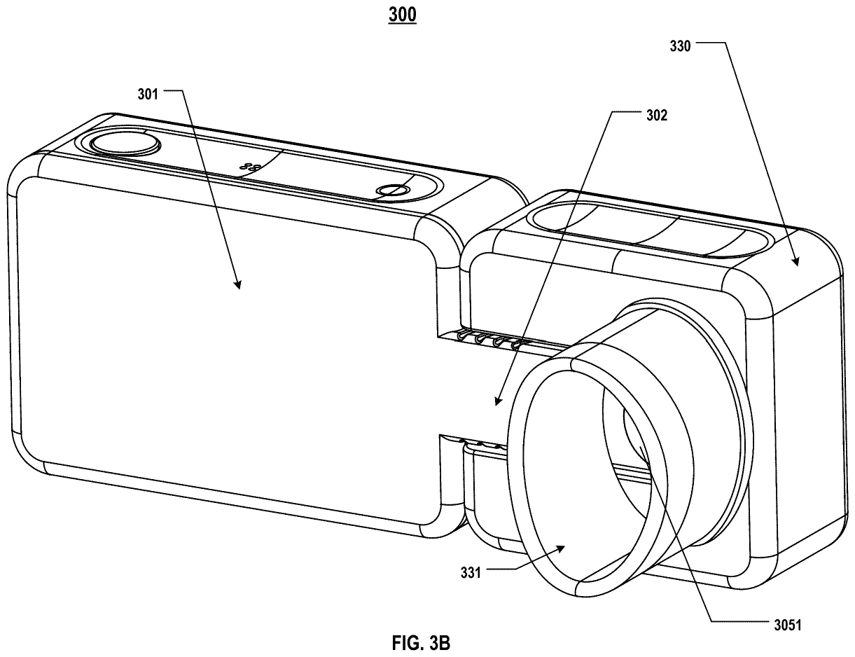

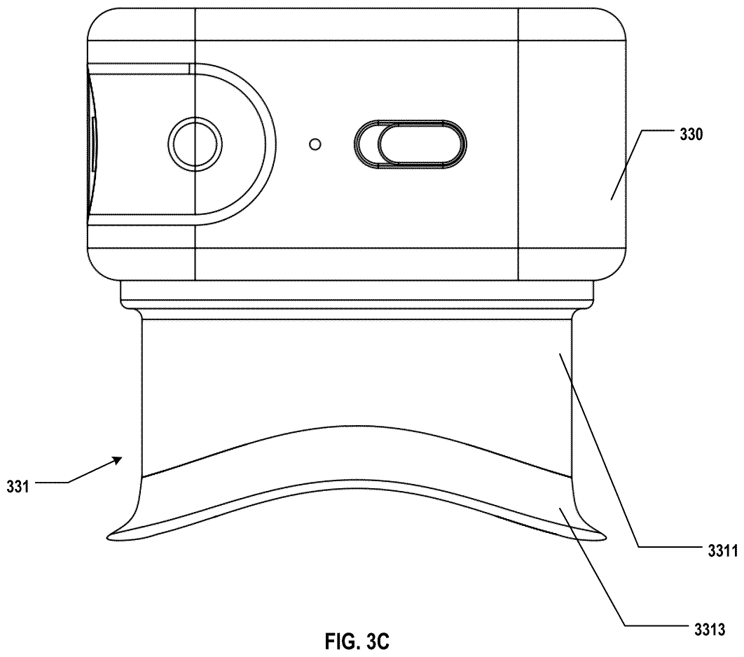

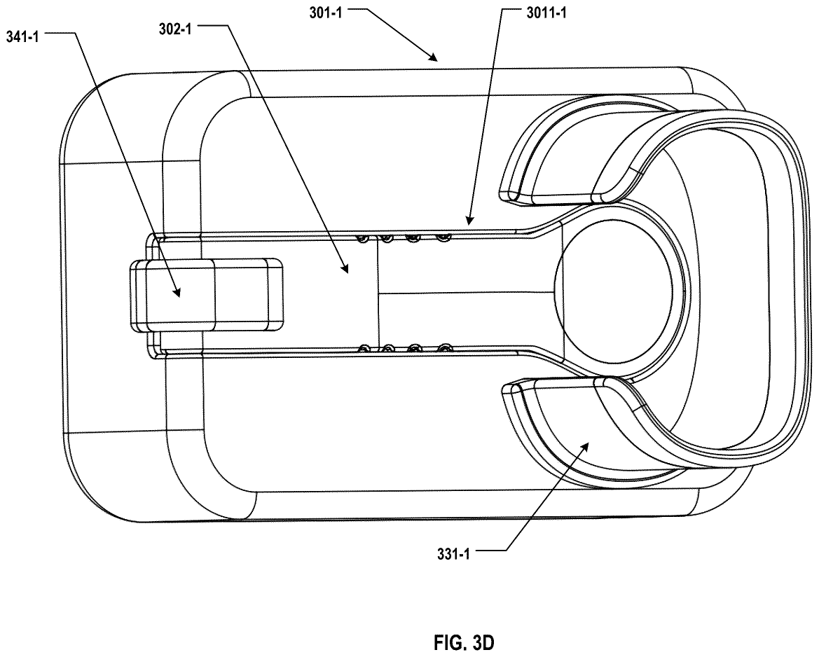

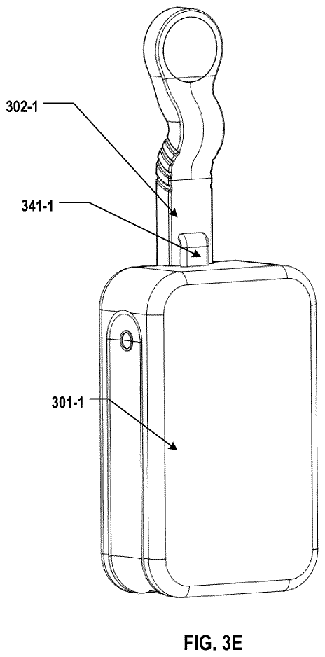

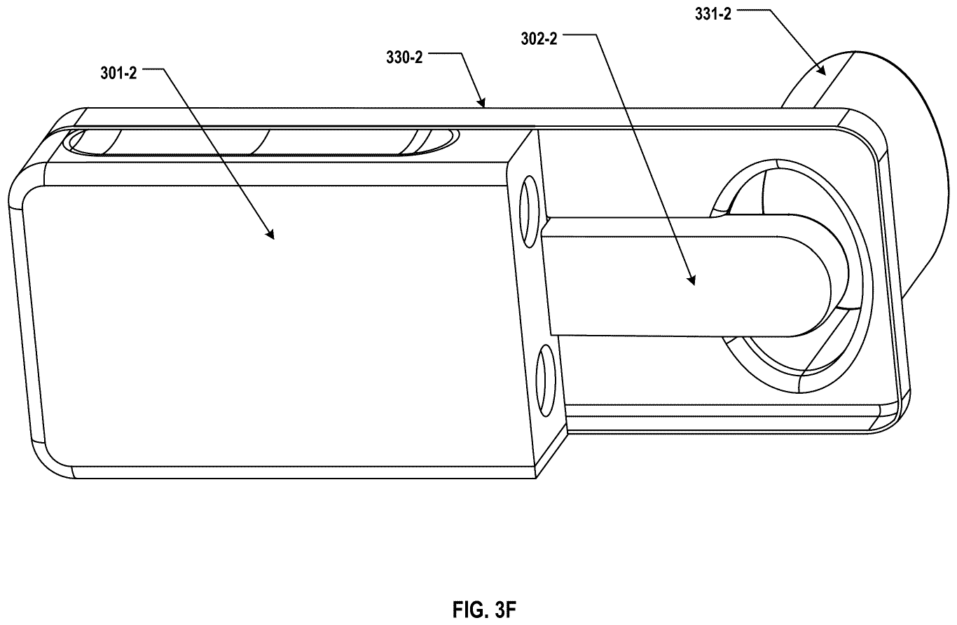

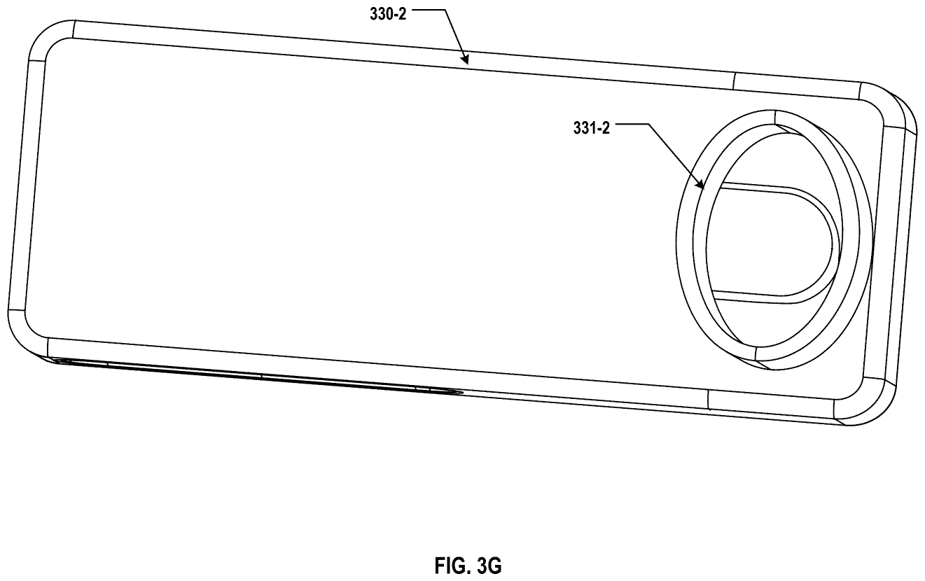

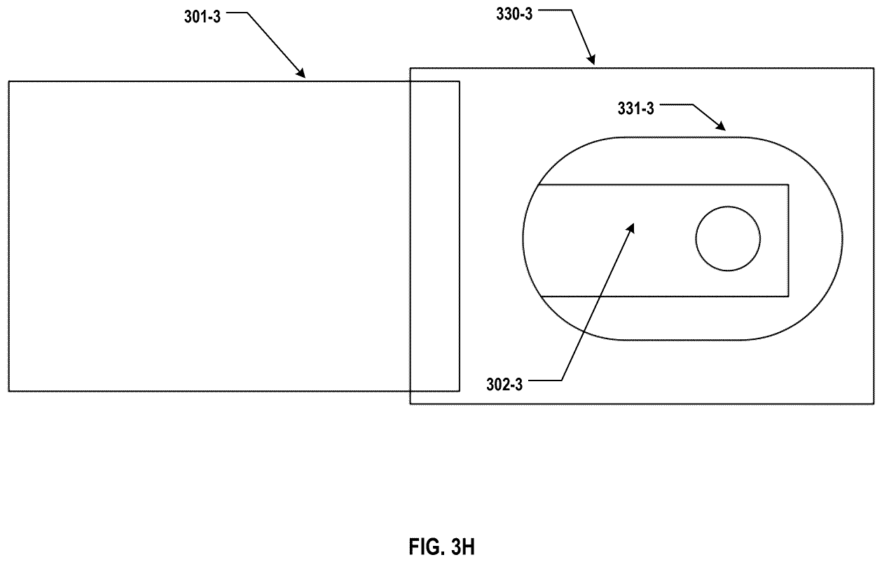

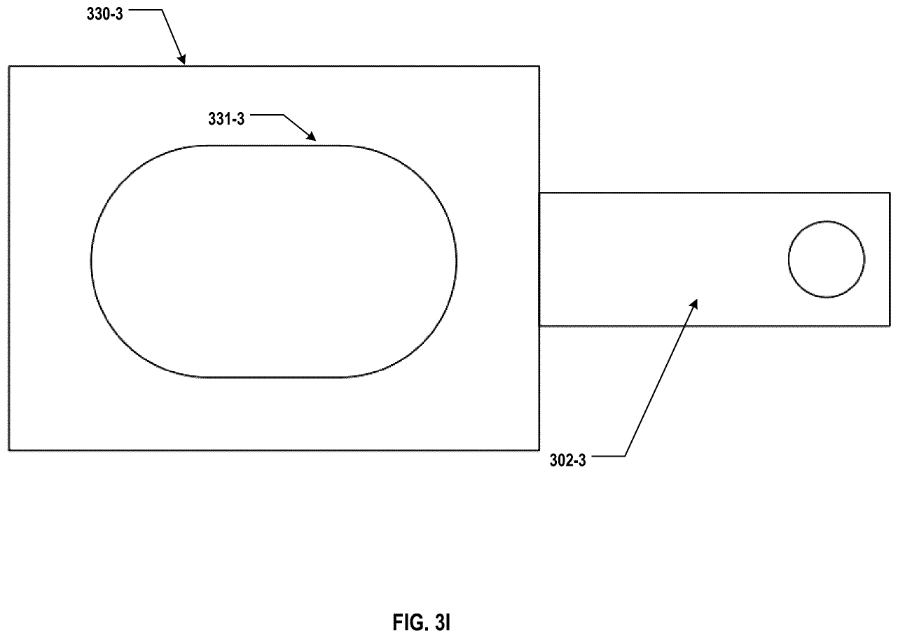

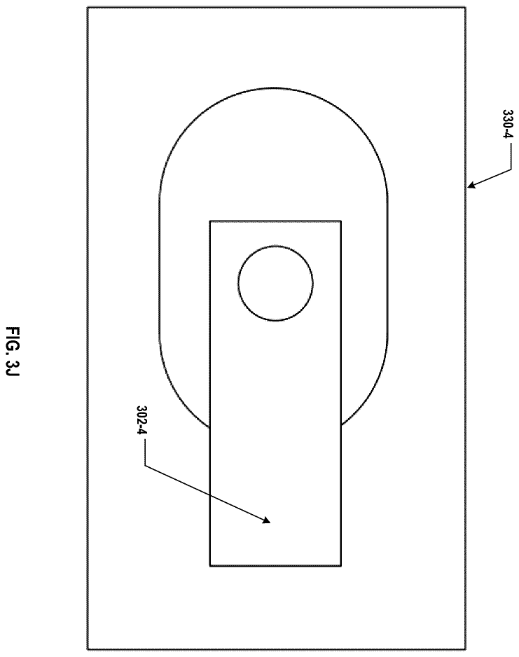

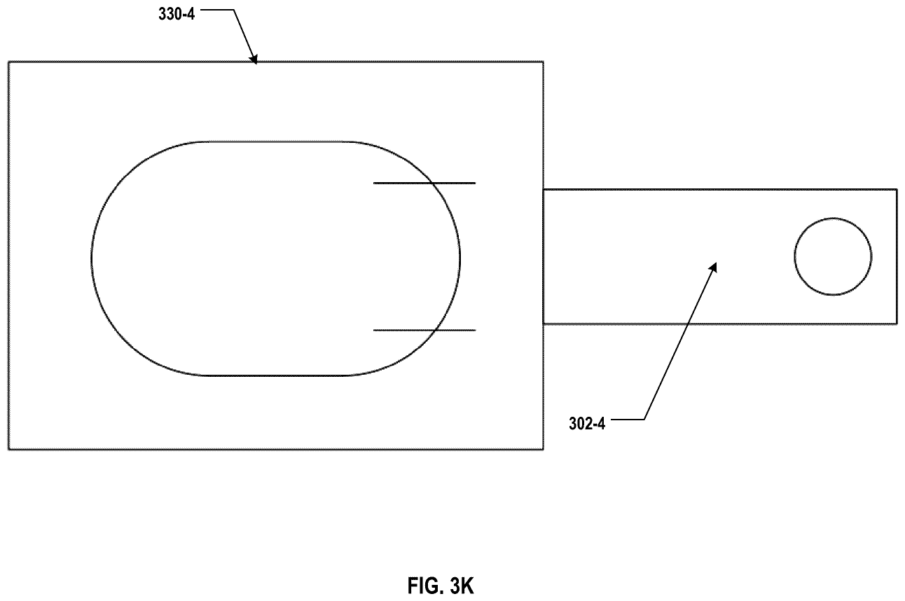

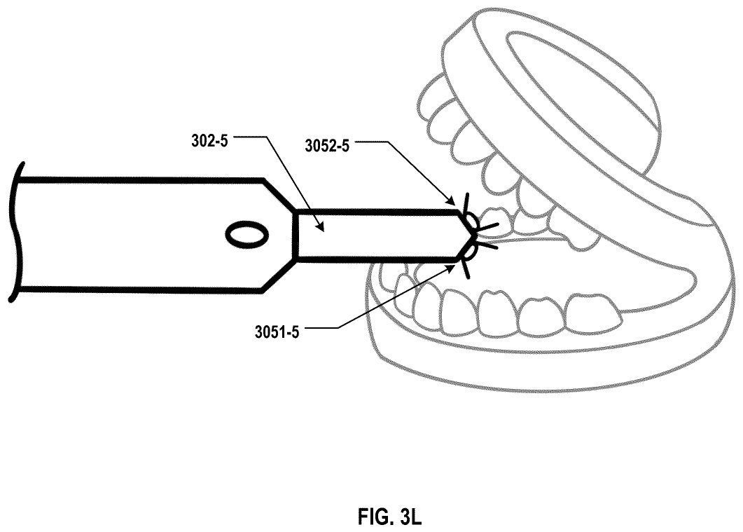

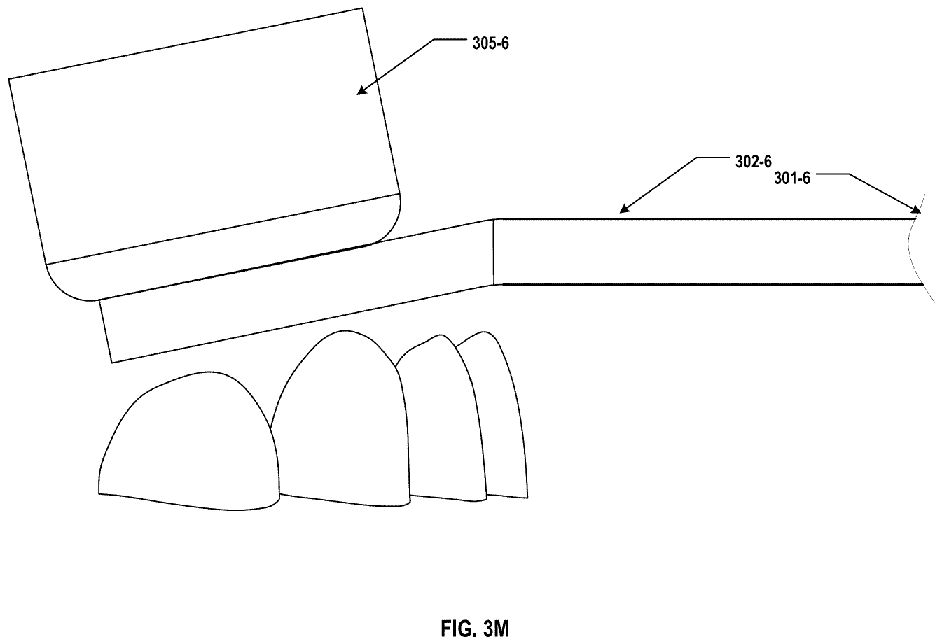

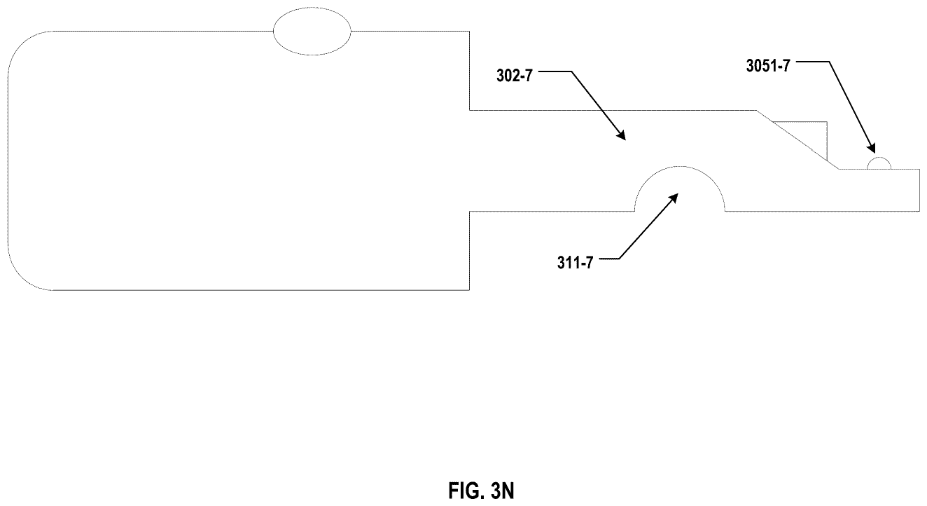

FIG. 1A illustrates a side view of an exemplary oral imaging device 100, according to some implementations of the present disclosure. As shown in FIG. 1A, oral imaging device 100 includes a body 101, a support arm 102 coupled to body 101, and a camera module 105 coupled to support arm 102. Body 101 can be a housing of a controller, a battery, or other electronic components that control the operations of oral imaging device 100. In some implementations, body 101 can be a rounded handle, enabling the user or dentist to grip and adjust the angle or position of camera module 105 while capturing images of a patient's oral cavity or teeth. The shape of body 101 is not limited to circle; it can also be square, rectangular, hexagonal, or any other shape that is easy for the user or dentist to hold and operate. In some implementations, there is a display module (e.g., display module 351 in FIG. 3A) on body 101, and the orientation of the display module is opposite to that of camera module 105. For instance, when camera module 105 faces the front surface, the display module faces the rear surface. The display module is used to display the image obtained by camera module 105, enabling the user to monitor and adjust the positioning during oral cavity photography, thereby ensuring optimal results. In some implementations, a processer of the controller in body 101 can analyze the captured images, and display module 105 can present the captured images and/or the analysis result to guide real-time adjustments.

Support arm 102 is coupled to body 101. In some implementations, support arm 102 is detachably attached to body 101, so that it can be removed from body 101 for replacement, cleansing, or storage purposes. Support arm 102 may include an external end 103 coupled to body 101 and an intra-oral end 104 coupled to camera module 105. It is noted that the dividing line between the two ends as shown in FIG. 1A is for illustration purpose only and does not indicate the actual size or location of the line. In some implementations, a thickness of camera module 105 is equal to or less than 12 mm, for example, 12 mm, 10 mm, 8 mm, 6 mm, etc. Such a slim design allows camera module 105 to be set lower than the uppermost part of the lower dental arch inside the patient's oral cavity, thereby increasing the distance between the camera and the upper dental arch and allowing capture of larger or wider images of the upper dental arch. Similarly, the slim design allows camera module 105 to be set higher than the lowermost part of the upper dental arch inside the patient's oral cavity, thereby increasing the distance between the camera and the lower dental arch and allowing capture of larger or wider images of the lower dental arch.

In some implementations, as shown in FIG. 1A, support arm 102 includes a first guide structure 111 on intra-oral end 104. Referring to FIG. 1B, support arm 102 can be stabilized by first guide structure 111 when oral imaging device 100 rests on a dental arch (e.g., a lower dental arch 153) while an aperture 1053 of camera 1051 of camera module 105 faces toward the other dental arch (e.g., an upper dental arch 151). It is worth noting that support arm 102 can also be stabilized by first guide structure 111 when oral imaging device 100 rests on upper dental arch 151, with camera 1051 of camera module 105 facing toward lower dental arch 153, as shown in FIG. 1C. With such arrangements, camera 1051 can capture images of at least part or entirety of the dental arch (e.g., the upper or lower dental arch). To perform a scanning operation to capture images of the dental arch, as shown in FIG. 1B, a user or dentist may have the patient open their mouth to approximately 20° to 45°, and then insert camera 1051 into the patient's oral cavity. First guide structure 111 allows camera module 105 to rest on lower dental arch 153 so that camera 1051 may be set lower than the uppermost part of lower dental arch 153 inside the patient's oral cavity. This setup sinks the camera into the inner part of the oral cavity relative to the shooting object and increases the distance between upper dental arch 151 and camera 1051, enabling camera 1051 to capture a full view (e.g., a panoramic view) of the entire upper dental arch 151 in a single image. The angle of opening the patient's oral cavity during image capture can also be reduced in comparison to the case where a conventional oral imaging device is used.

In some implementations, oral imaging device 100 may rest on an anterior tooth of lower dental arch 153 via first guide structure 111 to capture an occlusal surface of upper dental arch 151. In some implementations, first guide structure 111 may be used to control the depth at which camera module 105 is inserted into the oral cavity or to maintain a distance between camera 1051 and the corresponding dental arch (e.g., lower dental arch 153 or upper dental arch 151) to be captured, allowing for a full view of the dental arch being captured in a single image. In some implementations, first guide structure 111 includes at least one of a concave, a convex, or a bending structure.

For example, as shown in FIG. 1A, first guide structure 111 is a bending structure. In some implementations, as shown in FIG. 1D, an angle of the bending of first guide structure 111 may be designed to accommodate the patient's dental arch. For example, the angle of the bending (inward) of first guide structure 111 may be between 15° and 60°. In some implementations, the bending structure allows camera module 105 to be set lower than the uppermost part of the lower dental arch by 10 mm or more, for example, 10 mm, 12 mm, 14 mm, etc. In some implementations, the support arm is bent, but the optical axis of the camera can still remain perpendicular to the extension direction of the body. That is, the camera is positioned in the lower part of the oral cavity, angled upward to capture the upper dental arch.

In some implementations, the concave includes at least one of dot holes or trenches. In some implementations, the convex includes at least one of dot protrusions or strip protrusions. In some implementations, a depth of the concave is equal to or less than 3.5 mm, for example, 3.5 mm, 3.0 mm, 2.5 mm, 2.0 mm, 1.5 mm, 1.0 mm, etc. This allows the support arm to rest securely on the dental arch using the guide structure, serving as a fulcrum for a seesaw-like rotation to follow the designated trajectory. In some implementations, a width of the concave is equal to or less than 8 mm, for example, 8 mm, 6 mm, 4 mm, etc. In some implementations, a surface of first guide structure 111 contacting the upper surface of the dental arch (e.g., upper dental arch 151 or lower dental arch 153) is smooth in a width direction. Thus, when pressure is applied to first guide structure 111 by, e.g., patient's biting, the smooth surface tends to equally disperse the pressure along the width direction, and consequently camera module 105 is able to resist lateral movement on the dental arch. It is noted that support arm 102 extends away from body 101 in a length direction, and a rear surface 1042 and a front surface 1041 are opposite in a thickness direction.

In some implementations, as shown in FIG. 1A, support arm 102 may further include a second guide structure 121. Referring to FIG. 1E, support arm 102 can be stabilized by second guide structure 121 when oral imaging device rests on its side on a dental arch (e.g., lower dental arch 153) or between two dental arches (i.e., dental arches 151 and 153). In this way, camera 1051 may face toward inner surfaces of at least a portion of upper dental arch 151 or a portion of lower dental arch 153. To perform a scanning operation to capture images of the dental arch, as illustrated in FIG. 1E, the user or dentist inserts camera 1051 into the patient's oral cavity and has the patient bite down on support arm 102 via second guide structure 121. Second guide structure 121 limits the position of camera module 105 which either rests on lower dental arch 153 or is positioned roughly between the two dental arches, allowing camera 1051 to capture images of the inner surfaces of at least part of upper dental arch 151 or part of lower dental arch 153. The patient's teeth biting on second guide structure 121 may act as a pivot or fulcrum, enabling camera 1051 to rotate when the user or dentist moves body 101 (e.g., the handle) up and down. This rotation allows the user or dentist to capture images from different angles of the inner surfaces of the upper and lower dental arches as needed. For example, to get a better view of the molar teeth, the user or dentist can rotate camera 1051 to a position away from the molar teeth to capture a full image of the molar teeth.

Additionally, second guide structure 121 can be used to control the insertion depth of camera module 105 into the patient's oral cavity. For example, second guide structure 121 may include several stepped protrusions or trenches (e.g., two to eight steps) to allow the user or dentist to adjust the camera to the optimal position for capturing images of the inner surfaces of the dental arch according to different sizes of patients'oral cavity. In some implementations, second guide structure 121 includes at least one of a concave or a convex, as shown in FIGS. 1A and 1E. In some implementations, the concave includes at least one of dot holes or trenches. In some implementations, the convex includes at least one of dot protrusions or strip protrusions. In some implementations, the depth of the concave is equal to or less than 2 mm, for example, 2.0 mm, 1.5 mm, 1.0 mm, 0.5 mm, etc. In some implementations, the width of the concave is equal to or less than 8 mm, for example, 8 mm, 6 mm, 4 mm, 2 mm, etc. The width of support arm 102 is equal to or less than 20 mm, for example, 20 mm, 18 mm, 16 mm, 14 mm, 12 mm, 10 mm, 8 mm, etc. This allows the user to open the mouth less widely, thereby reducing the distance between the upper and lower teeth, and allowing simultaneous capture of the inner surfaces of the upper and lower teeth. In some implementations, the concave may include an inclined groove. The inclined groove is tiled with the angle that aligns with the posture of the support arm as it extends into the oral cavity to capture the inner surfaces. In some implementations, the inclined groove is formed at both sides of the support arm. In some implementations, the surface of second guide structure 121 contacting the upper surface of the dental arch (e.g., upper dental arch 151 or lower dental arch 153) is smooth in a thickness direction. Thus, when pressure is applied to second guide structure 121 by, e.g., patent's biting, the smooth surface tends to equally disperse the pressure along the thickness direction, and consequently camera module 105 is able to resist lateral movement on the dental arch.

In some implementations, as shown in FIG. 1A, support arm 102 may further include a third guide structure 131. Referring to FIG. 1F, support arm 102 can be stabilized by third guide structure 131 when oral imaging device 100 rests on its side on a dental arch (e.g., lower dental arch 153, upper dental arch 151, or both) while camera 1051 faces toward the outer surface of at least a portion of upper dental arch 151 and lower dental arch 153. In some implementations, referring to FIG. 1G, support arm 102 can also be stabilized by third guide structure 131 when oral imaging device 100 rests on outer surfaces of lower dental arch 151 or the patient's jaw, with camera 1051 facing toward outer surfaces of at least a portion of upper dental arch 151 or a portion of lower dental arch 153. With such arrangements, camera 1051 can capture images of outer surfaces of at least part or entirety of dental arch (i.e., the upper or lower dental arch). To perform a scanning operation to capture images of the dental arch, as shown in FIG. 1F, a user or dentist may have the patient close their oral cavity, and then rest oral imaging device 100 on patient's outer surfaces of the teeth via third guide structure 131. Third guide structure 131 allows the support arm to be stabilized when the oral imaging device rests on the same side as the first dental arch or the second dental arch, in order to capture at least a portion of the first dental arch or a portion of the second dental arch.

As shown in FIGS. 1F and 1G, third guide structure 131 may lift camera 1051 away from the outer surface of the first dental arch or the second dental arch so that there is a threshold distance between the camera and the outer surface of the first dental arch or the second dental arch, which helps capturing better views of the outer surface of the dental arch. Third guide structure 131 may be used to maintain a distance between camera 1051 and the tooth surface when oral imaging device 100 rests on the patient's dental arch, jaw, gum, or teeth, and thus avoids obstructing the camera's field of view like a lens hood would. By incorporating a bending or protruding structure on the front surface, the camera remains unobstructed from the tooth surface. This design is particularly suitable for wide-angle cameras. For example, third guide structure 131 may be closer to the first dental arch than camera 1051 when camera 1051 rests on the second dental arch. In some implementations, third guide structure 131 includes at least one of a concave, a convex, or a bending structure. For example, as shown in FIGS. 1F-1G, third guide structure 131 is a bending structure. In some implementations, as shown in FIG. 1D, an angle of the bending (outward) of third guide structure 131 may be between 300° and 345° In some implementations, third guide structure 131 may lift camera 1051 away from the outer surface of the first dental arch or the second dental arch for a threshold distance, thus creating a space between the camera and the outer surface of the first dental arch or the second dental arch. The distance can be equal to or more than 8 mm, for example, 8 mm, 10 mm, 12 mm, 14 mm, etc. In some implementations, a portion of third guide structure 131 has at least one rounded surface in contact with the first dental arch or the second dental arch so that the user or dentist may easily adjust the angle or position of the camera module when capturing the outer surface of the dental arch.

In some implementations, as shown in FIG. 1A, first guide structure 111 is located on a rear surface 1042 of support arm 102, second guide structure 121 is located on a side surface 1043 of support arm 102, and third guide structure 131 is located on a front surface 1041 of support arm 102. It is noted that front surface 1041 faces the same or partially similar direction as camera 1051 of camera module 105 (i.e., an aperture of the camera) faces, whereas rear surface 1042 faces an opposite or at least partially opposite direction to camera 1051 of camera module 105. Side surface 1043 connects front surface 1041 and rear surface 1042. In some implementations, side surface 1043 may include a raised portion, and second guide structure 121 is located on the uppermost part of the raised portion of side surface 1043. Additionally, in some implementations, both side surfaces 1043 (i.e., the right and left side surfaces) may include raised portions, with the corresponding second guide structures situated on each side. In some implementations, rear surface 1042 and front surface 1041 are parallel to each other.

Furthermore, as shown in FIG. 1D, first guide structure 111 (e.g., the concave or bending structure) on the rear surface of the support arm can restrict the angle of the camera module by abutting against the inner and/or outer side of the teeth of the lower dental arch (or upper dental arch) through the concave. The camera's optical axis is set as perpendicular as possible to the occlusal surface, or at least between 60° and 90° during image capture, to avoid excessive shooting angles. Such excessive angles may block the view of the pits and grooves of the occlusal surface, preventing the entire occlusal surface from being captured in a single view. The optical axis of the camera can be slightly adjusted to tilt towards the inner side of the teeth in order to capture the corresponding inner side when capturing the occlusal surface. In the depth direction of the oral cavity, the tilt angle of the camera module can be adjusted according to the angle between the camera optical axis and the occlusal surface. In the lateral direction (i.e., left and right direction) of the oral cavity, the rear surface (except for the guide structure) has a flat or smooth surface, so that the rear surface contacting the teeth is relatively fixed and evenly placed and may not deviate in the lateral direction, thereby improving the stability of the support arm during the operation. That is, the distance between the camera and either of the left and right occlusal surfaces is roughly the same.

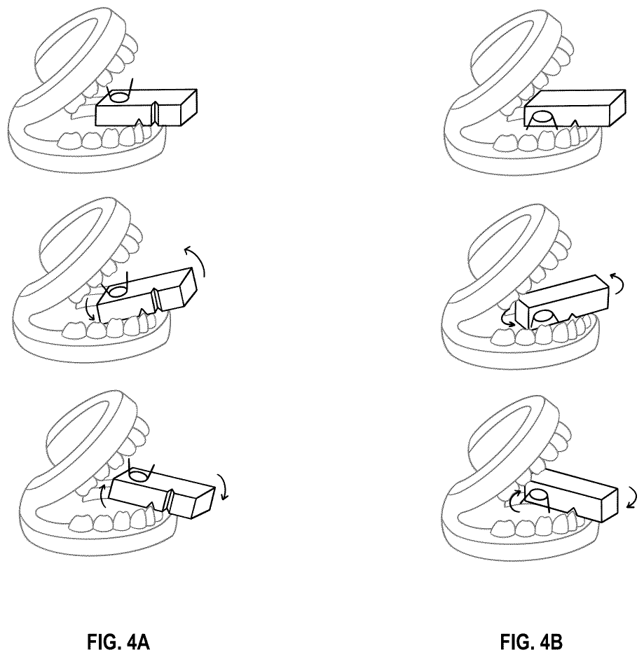

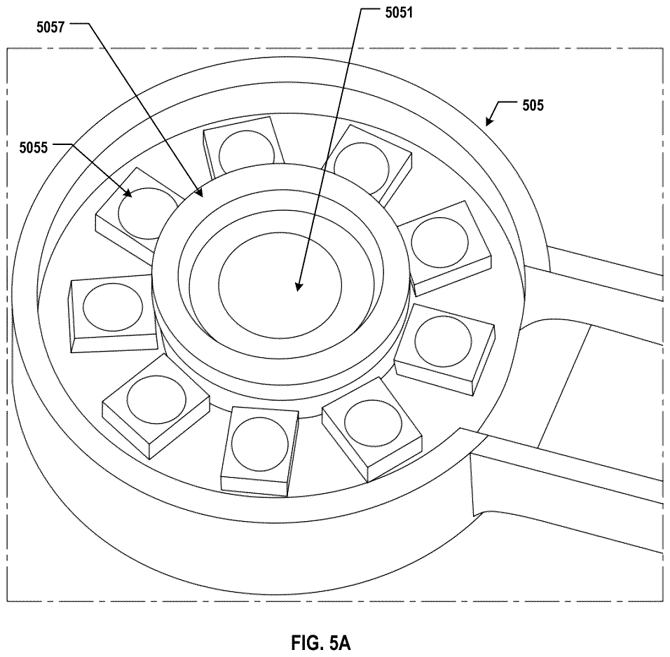

In some implementations, as shown in FIG. 4A, the camera module (including camera module 105 and/or any other camera modules disclosed herein) may move along a first trajectory. In particular, the camera module is configured to move along the first trajectory when the support arm is stabilized by the first guide structure and the oral imaging device rests on the lower dental arch or the upper dental arch, and captures at least an image including at least a portion of the upper dental arch or at least a portion of the lower dental arch. In some implementations, camera 1051 may capture at least 30% of the lower dental arch or the upper dental arch in a single image. In some implantations, the user or dentist may capture one to three images to have a full view of the entire lower dental arch or the upper dental arch. The multiple images may be combined by the user or dentist manually, or automatically by accompanying software. In some implementations, when the camera module rests on the dental arch using the first guide structure, the first guide structure acts as a pivot or fulcrum, enabling the camera to rotate vertically when the user or dentist pushes the body (e.g., the handle) up and down.

In some implementations, as shown in FIG. 4B, the camera module (including camera module 105 and/or any other camera modules disclosed herein) may move along a second trajectory. In particular, the camera module is configured to move along the second trajectory when the support arm is stabilized by the second guide structure and the oral imaging device rests on the lower dental arch or the upper dental arch, and captures at least an image including at least a portion of the upper dental arch and at least a portion of the lower dental arch. In some implementations, when the camera module rests on the dental arch using the second guide structure, the second guide structure acts as a pivot or fulcrum, enabling the camera to rotate laterally when the user or dentist pushes the body left or right. In some implementations, a plane of the second trajectory is different from that of the first trajectory. In some implementations, a plane of the second trajectory can be a lateral plane, as illustrated in FIG. 4B, while a plane of the first trajectory can be a vertical plane, as illustrated in FIG. 4A. That is, in such cases, the plane of the second trajectory and the plane of the first trajectory are perpendicular to each other.

In some implementations, referring to FIG. 1A, the camera (including camera 1051 and/or other camera disclosed herein) can be a wide-angle camera. For example, a field of view (FOV) of camera 1051 is equal to or larger than 140°, for example, 140°, 145°, 150°, 157°, 160°, 163°, 170°, 172.5°, 177.8°, 180°, etc., so that oral imaging device 100 may be capable of capturing a full view of the dental arch in a single image when inserting the oral imaging device into the patient's oral cavity. For example, when the distance between the camera and the object (e.g., the upper dental arch) to be captured is set to 20 mm, the shorter side of the camera's captured image is not less than 60 mm (with the diagonal being not less than 100 mm) so that the entire view of the dental arch can be captured in one shot. Under this distance setup, the FOV is approximately 140° or more.

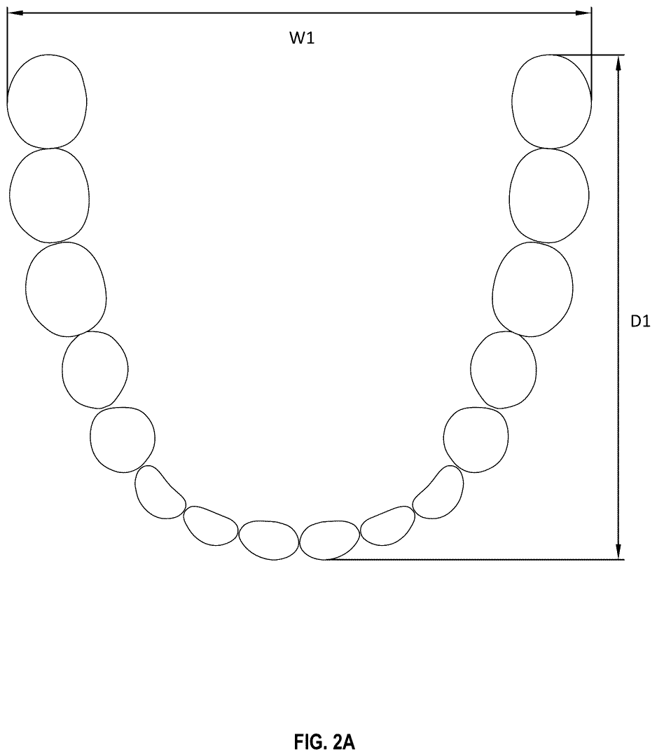

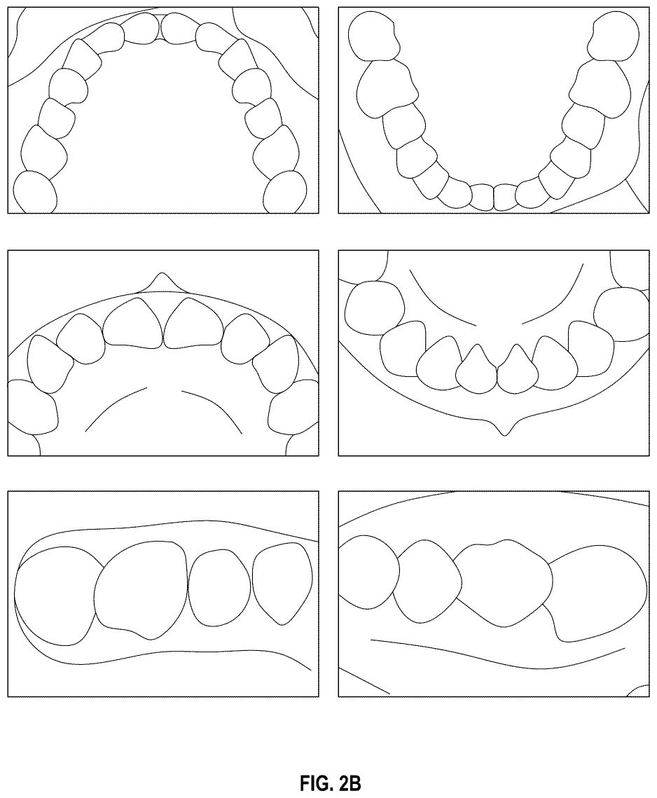

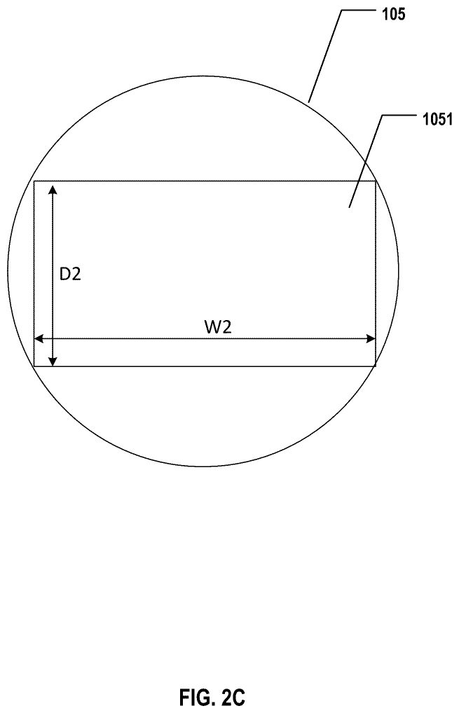

In some implementations, camera 1051 has a rectangular FOV, as shown in FIG. 2C. A shorter side of the rectangular FOV (i.e., D2) is parallel to a length direction of support arm 102 (e.g., a direction along which support arm 102 extends away from body 101). A longer side of the rectangular FOV (i.e., W2) is parallel to a width direction of support arm 102 (e.g., a lateral direction of support arm 102). In some implementations, the ratio between W2 and D2 is 4:3 or 16:9. In some implementations, an angle between an optical axis of camera 1051 and an occlusal surface of a patient's oral cavity is between 60° and 90° when oral imaging device 100 rests on the dental arch (e.g., upper or lower dental arch). In some implementations, an angle between the optical axis of camera 1051 and a length direction of body 101 is between 40° and 90°. As shown in FIG. 2A, a dental arch has a depth D1 ranged from edges of molars to edges of incisors and a width W1 ranged from edges of molars on both sides. That is, the shorter side of the rectangular FOV is parallel to the depth D1 inside the patient's oral cavity while the longer side of the rectangular FOV is parallel to the width H1, so that the oral imaging device can effectively capture a full view of the dental arch.

FIG. 2B illustrates commonly used oral images, for example, full views of upper or lower dental arch, front views of upper or lower dental arch, and partial views of occlusal surfaces of molars. By using the above arrangements, the user or dentist may adjust the angle and position of the camera inside the patient's oral cavity to capture these images accordingly.

Referring to FIG. 1H, lengths H1 and H2 represent the respective lengths of the upper dental arch and lower dental arch. Take the average length of the dental arch of adults as an example and assume H1=H2=50 mm. According to the previous descriptions, the camera's FOV angle is not less than 140 °, i.e., γ≥140°. In addition, taking the upper occlusal surface as an example, ideally, the lens of camera 1051 is parallel to the upper dental arch, and camera 1051 is located approximately on the midline of the upper dental arch, making it easier to obtain a full view of the upper dental arch in a single image. When the angle of oral opening is β and the angle of camera inclination (i.e., an angle at which the camera tilts up relative to the length direction of the support arm) is α, it can be concluded that β=α. According to the above, the range of mouth opening angles for adults is between 30° and 45°, while that for children is between 20° and 30°. Therefore, the tilt angle α of camera 1051 is preferred to be between 25° and 35°. Combined with the design of the fulcrum (i.e., the guide structure), which has a fine-tuning function for the user or dentist, the oral imaging device according to the present disclosure can capture the entire occlusal surface and some inner surfaces of the dental arch in one single image.

FIG. 3A illustrates another exemplary embodiment of the present disclosure. It is noted that similar features, structures or characteristics described in connection with FIG. 1A may also be incorporated into embodiments described in connection with FIG. 3A wherever applicable. As shown in FIG. 3A, oral imaging device 300 includes a body 301, a support arm 302 coupled to body 301, and a camera module 305 coupled to support arm 302. Body 301 can be a housing of a controller, a battery, or other electronic components that control the operations of oral imaging device 300. In some implementations, body 301 can be a handle with a shape of rectangular box, enabling the user or dentist to grip and adjust the angle or position of camera module 305 while scanning a patient's oral cavity or teeth. The shape of body 301 is not limited to the rectangular box; it can also be square, hexagonal, pillar, or any other shape that is easy for the user or dentist to hold and operate. Support arm 302 is coupled to body 301. In some implementations, support arm 302 is detachably attached to body 301 so that it can be removed from body 301 or fixed on body 301. By having the detachable feature, it can realize both intra-oral scanning and extra-oral scanning in a single oral imaging device. That is, an intra-oral and extra-oral two-in-one oral imaging device is realized. The definition of intra-oral and extra-oral is distinguished by whether the camera is inserted into the oral cavity (i.e., whether the camera extends beyond the inner surface of the anterior teeth). In some implementations, support arm 302 can also be a part of body 301. Support arm 302 may include an external end 303 coupled to body 301 and an intra-oral end 304 coupled to camera module 305.

In some implementations, as shown in FIG. 3A, support arm 302 includes a first guide structure 311. Referring to FIG. 3B, support arm 302 can be stabilized by first guide structure 311 when oral imaging device 300 rests on one dental arch while an aperture of camera 3051 of camera module 305 faces toward the other dental arch. It is worth noting that support arm 302 can also be stabilized by first guide structure 311 when oral imaging device 300 rests on the upper dental arch, with camera 3051 of camera module 305 facing the lower dental arch. The spatial arrangements are substantially the same as those described in the embodiments illustrated in FIGS. 1A-1C. With such arrangements, camera 3051 can capture images of at least part or entirety of a dental arch (e.g., the upper dental arch or the lower dental arch). To perform a scanning operation to capture images of the dental arch, a user or dentist may have the patient open their oral cavity for approximately 20° to 45°, and then insert camera 3051 into the patient's oral cavity. First guide structure 311 allows camera module 305 to rest on the lower dental arch so that camera 3051 may be set lower than the uppermost part of the lower dental arch inside the patient's oral cavity. This setup sinks the camera into the lower part of the oral cavity relative to the image capturing device and increases the distance between the upper dental arch and camera 3051, thereby enabling camera 3051 to capture a full view (e.g., a panoramic view) of the entire upper dental arch in a single image. The opening angle of the patient's oral cavity during the image capturing can also be reduced.

In some implementations, oral imaging device 300 may rest on an anterior tooth of the lower dental arch via first guide structure 311 to capture an occlusal surface of the upper dental arch. In some implementations, first guide structure 311 may be used to control the depth at which camera module 305 is inserted into the oral cavity or to maintain a distance between camera 3051 and the corresponding dental arch to be captured, thereby enabling a full view of the dental arch to be captured in a single image. In some implementations, first guide structure 311 includes at least one of a concave or a convex, as shown in FIG. 3A. In some implementations, as shown in FIG. 3A, an angle of first guide structure 311 may be designed to cause oral imaging device 300 to attach to patient's dental arch. For example, the angle of the concave or bending (inward) of first guide structure 311 may be between 15° and 60°. In some implementations, the concave includes at least one of dot holes or trenches. In some implementations, the convex includes at least one of dot protrusions or strip protrusions. In some implementations, the depth of the concave is equal to or less than 3.5 mm. In some implementations, the width of the concave is equal to or less than 8 mm. In some implementations, a surface of first guide structure 311 contacting the upper surface of the dental arch is smooth in a width direction. Thus, when pressure is applied to first guide structure 311 by, e.g., patient's biting, the smooth surface tends to equally disperse the pressure along the width direction, and consequently camera module 305 is able to resist lateral movement on the dental arch. It is noted that support arm 302 extends away from body 301 in a length direction, and a rear surface (not shown) and a front surface 3041 are opposite in a thickness direction.

In some implementations, as shown in FIG. 3A, support arm 302 may further include a second guide structure 321. Referring to FIG. 3A, support arm 302 can be stabilized by second guide structure 321 when oral imaging device 300 rests on one of the dental arches or between two dental arches while camera 3051 faces toward inner surfaces of at least a portion of the upper dental arch or the lower dental arch. To perform a scanning operation to capture images of the dental arch, as illustrated in FIG. 3A, the user or dentist may insert camera 3051 into the patient's oral cavity and have the patient bite down on support arm 302 via second guide structure 321. This guide structure 321 supports camera module 305, either resting on the lower dental arch or being positioned approximately between the two dental arches, allowing camera 3051 to capture images of the inner surfaces of at least part of the upper dental arch or the lower dental arch. The patient's teeth biting on second guide structure 321 may act as a pivot or fulcrum, enabling camera 3051 to rotate when the user or dentist moves body 301 (e.g., the handle) up and down. This rotation allows the user or dentist to capture images from different angles of the inner surfaces of the upper and lower dental arches as needed. For example, to get a better view of the molar teeth, the user or dentist can rotate camera 3051 to a position away from the molar teeth to capture a full image of the molar teeth.

Additionally, second guide structure 321 can be used to control the insertion depth of camera module 305 into the patient's oral cavity. For example, second guide structure 321 may include several stepped protrusions or trenches (e.g., three to eight steps), as illustrated in FIG. 3A, to allow the user or dentist to adjust the camera to the optimal position for capturing images of the inner surfaces of the dental arch according to different sizes of patients'oral cavity. In some implementations, second guide structure 321 includes at least one of a concave or a convex, as shown in FIG. 3A. In some implementations, the concave includes at least one of dot holes or trenches. In some implementations, the convex includes at least one of dot protrusions or strip protrusions. In some implementations, the depth of the concave is equal to or less than 3.5 mm. In some implementations, the width of the concave is equal to or less than 8 mm. In some implementations, the surface of second guide structure 321 contacting the upper surface of the dental arch is smooth in a thickness direction. Thus, when pressure is applied to second guide structure 121 by, e.g., patent's biting, the smooth surface tends to equally disperse the pressure along the thickness direction, and consequently camera module 305 is able to resist lateral movement on the dental arch.

In some implementations, as shown in FIG. 3A, first guide structure 311 is located on a rear surface (e.g., a surface opposite to front surface 3041) of support arm 302, and second guide structure 321 is located on a side surface 3043 of support arm 302. It is noted that front surface 3041 faces the same or substantially the same direction as camera 3051 (i.e., a direction which an aperture of the camera faces), whereas the rear surface faces the opposite or at least partially opposite direction to camera 3051. Side surface 3043 is connected between front surface 3041 and the rear surface. In some implementations, the rear surface and front surface 3041 are parallel to each other.

Furthermore, similar to the embodiments illustrated in FIG. 1D, as shown in FIG. 3A, first guide structure 311 (e.g., the concave or bending structure) on the rear surface of the support arm can restrict the angle of the camera module by abutting against the inner and/or outer side of the teeth of the lower dental arch (or upper dental arch) through the concave. To avoid difficult shooting angles during the imaging capturing process, the camera's optical axis is set to be as perpendicular as possible to the occlusal surface, or at least the angle between the optical axis and the occlusal surface is set to be between 60° and 90°; otherwise, the view of the pits and grooves of the occlusal surface may be blocked, and as a result, the entire occlusal surface cannot be captured in a single view. The optical axis of the camera can be slightly adjusted to tilt towards the inner side of the teeth to capture the corresponding inner side while also capturing the occlusal surface. In the depth direction of the oral cavity, the tilt angle of the camera module can be adjusted according to the angle between the camera optical axis and the occlusal surface. In the lateral direction (i.e., left or right) of the oral cavity, the rear surface (except for the guide structure) has a flat or smooth surface, so that the rear surface contacting the teeth is relatively fixed and evenly placed and may not deviate in the lateral direction. That is, the distance between the camera and either of the left and right occlusal surfaces is roughly the same.

Referring to FIG. 3B, oral imaging device 300 may further include a housing 330 and a mouth gag 331 coupled to housing 330. In some implementations, the gag is coupled to the housing or is a part of the housing. In some implementations, housing 330 can be detachably attached to support arm 302 and body 301. In some implementations, housing 330 is detachably coupled to body 301 on a first side of body 301, and support arm 302 extends away from the first side of body 301. In some implementations, housing 330 is sized to accommodate support arm 302, as illustrated in FIG. 3B.

In addition, mouth gag 331 includes at least an opening. The opening of mouth gag 331 corresponds to an aperture of camera 3051 to allow the camera to capture the image via the opening of mouth gag 331. In some implementations, the relative position between mouth gag 331 and support arm 302 is adjustable. For example, when the mouth gag is detached from the body, the relative position between the mouth gag and the support arm is changed. In another example, the support arm is rotatable via a shaft so that when the support arm is rotated outside of the housing, the relative position between the mouth gag and the support arm is changed. In some implementations, as shown in FIG. 3C, mouth gag 331 includes a main portion 3311 and an extension portion 3313. In some implementations, main portion 3311 is detachably coupled to housing 330, and extension portion 3313 is disposed on one end of mouth gag 331 away from housing 330. During the operation, the patient may bite on mouth gag 331, and extension portion 3313 may stop the teeth, spread the lip, and keep the patient's oral cavity in place so that when camera 3051 captures images of the outer surface of the teeth, the teeth will not move and the oral imaging device will be stabilized in position. In some implementations, mouth gag 331 is in the shape of a protective cover and not transparent. In addition, mouth gag 331 also has the function of concentrating light and blocking ambient light and maintaining the shooting distance from the camera to the outer surfaces of the teeth.

In some implementations, as shown in FIG. 3D, support arm 302-1 is rotatable via a shaft 341-1 coupled to body 301-1. Support arm 302-1 may be accommodated in a groove 3011-1 of body 301-1 when support arm 302-1 is folded (as shown in FIG. 3D), and support arm 302-1 extends away from body 301-1 when support arm 302-1 is unfolded (as shown in FIG. 3E).

In some implementations, as shown in FIGS. 3F and 3G, support arm 302-2 is coupled to body 301-2, and housing 330-2 is detachably coupled to body 301-2 and support arm 302-2 in a main surface of body 301-2, where the main surface is the largest surface of body 301-2. Housing 330-2 may be a shell-like shape with a mouth gag 331-2. Housing 330-2 includes a first side and a third side that opposite each other. An opening of hosing 330-2 is set on the first side, and the third side is used to connect body 301-2. This housing design has a simple structure and can obtain a larger imaging area of the patient's oral cavity with good connection stability, and the camera does not extend into the opening, or the camera can be set further away from the opening to obtain a larger shooting distance. In some implementations, the connection method between the housing and the body herein may include magnetic connection, bayonet connection, sleeve connection, or the like. In some implementations, the housing is magnetically attracted to the side of the body, which is convenient to connect and has a large magnetic attraction surface and makes the connection more stable. At the same time, the bending structure on the edge of the housing also has a good limiting effect. The housing can be attached and detached using planar magnetic attraction, with its edges secured to the edges of the body to restrict planar movement, providing a strong attachment.

In addition, when housing 330-2 is connected to body 301-2, the outer surface of housing 330-2 is flat with the surface of the body, so that the oral imaging device has a rectangular shape to be like a camera. The housing 330-2 is connected to the body and is used to cover or block the support arm and a portion of the camera, preventing cross contact or contaminants during public use. It can also provide certain protection for the camera or the support arm.

In some implementations, as shown in FIGS. 3H and 3I, housing 330-3 with mouth gag 331-3 attached thereto, is slidably coupled to body 301-3. Housing 330-3 may slide away from (as illustrated in FIG. 3H) or close to (as illustrated in FIG. 3I) body 301-3 in a length direction, and support arm 302-3 extends away from body 301-3 in the length direction.

In some implementations, as shown in FIGS. 3J and 3K, support arm 302-4 is detachably coupled to the body or housing 330-4. Based on the above arrangement, when the opening of the mouth gag is aligned with the camera, the oral imaging device is used for extra-oral image capturing; and when the opening of the mouth gag is offset from the camera, the camera and the support arm are exposed and protruding, which is suitable for intra-oral shooting. That is, an intra-oral and extra-oral two-in-one scanner can be realized.

In some implementations, as shown in FIG. 3L, more than one camera can be set on the support arm. For example, a first camera 3051-5 and a second camera 3052-5 can be coupled to support arm 302-5. An angle between an aperture of first camera 3051-5 and that of second camera 3052-5 may be between 130° and 160°, for example, 130°, 135°, 140°, 143°, 147.5°, 150°, 156.8°, or 160°.

In some implementations, as shown in FIG. 3M, camera module 305-6 can be set on support arm 302-6 with an inclination angle. For example, an angle between camera module 305-6 and body 301-6 or between camera module 305-6 and a portion of support arm 302-6 in contact with body 301-6 may be between 20° and 40°, for example, 20°, 25°, 27.5°, 30°, 33.8°, 38.4°, or 40°. This allows the camera to be positioned away from the object to be captured for a distance, and thus a full view of the object can be captured more easily.

In some implementations, as shown in FIG. 3N, the rear surface of support arm 302-7 is first guide structure 311-7 (e.g., a concave), and camera 3051-7 leans back relative to the front surface of support arm 302-7. The concave structure on the rear surface of the support arm can be placed on the incisor of the lower dental arch when shooting the upper dental arch, and placed on the incisor of the upper dental arch when shooting the lower dental arch. The concave structure not only keeps the camera further away from the object being captured, but also serves as a limiting function for its intended position. It also works in conjunction with teeth to restrict the depth of the camera's insertion into the oral cavity, thereby improving the overall functionality of the oral imaging device.

In some implementations, a guide structure (e.g., first guide structure 111, second guide structure 121, or other guide structures described herein) of the support arm can limit the position of the camera module relative to the oral cavity. The guide structure is used to control the depth at which camera module 105 is inserted into the oral cavity or to maintain a distance between camera 1051 and the corresponding dental arch (e.g., lower dental arch 153 or upper dental arch 151) to be imaged. Furthermore, the support arm may include side surfaces, and the guide structure and the camera module are disposed on the side surfaces. For instance, the guide structure and the camera module may be located on opposite sides, adjacent sides, or the same side of the support arm.

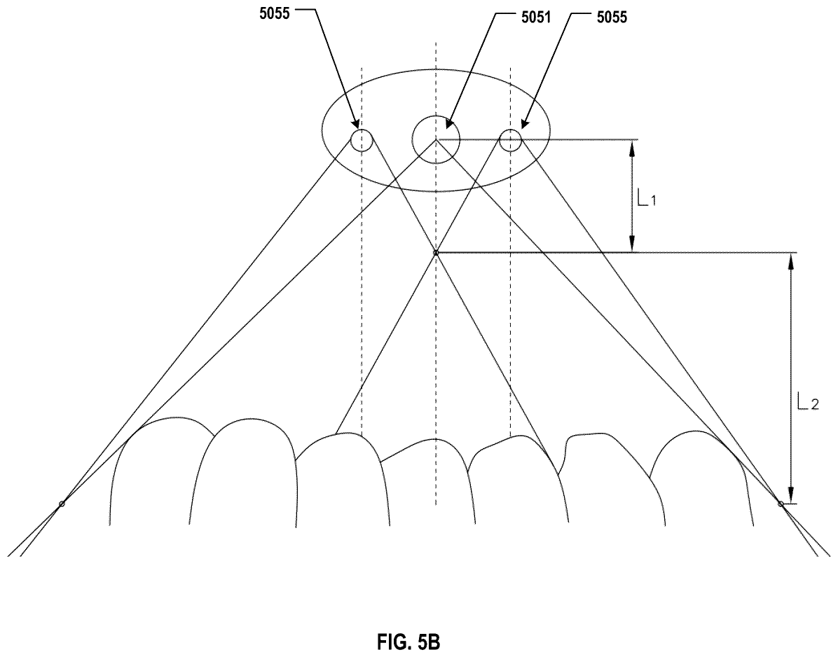

Referring to FIG. 5A, as mentioned above, the oral imaging device (e.g., oral imaging device 100 or 300) may include a camera module (for example, camera module 105, 305, 505, or other camera modules described herein). Camera module 505 may include a camera 5051, a lens hood 5057 surrounding camera 5051, and one or more lightings 5055 surrounding camera 5051 and lens hood 5057. In some implementations, each of lightings 5055 may generate a light including at least one of a white light, a blue light, a purple light, an ultraviolet (UV) light, or a red light. In some implementations, each of these lightings 5055 may generate a light including a wavelength in a range selected from at least one of the following ranges: 315 nm to 400 nm, 380 nm to 750 nm, 450 nm to 500 nm, or 750 nm to 1,500 nm. It is noted that the functions of the lightings are not limited to providing sufficient illumination, it can also be used to provide specific light wavelength that assists scanning or examining the corresponding objects. For example, a purple light provided by the lightings can be used to stimulate the fluorescent effect of bacterial plaque.

In some implementations, camera 5051 and lightings 5055 are arranged so that a distance L1 between camera 5051 and an intersection of light paths of the plurality of lightings 5055 is equal to or less than 10 mm, as shown in FIG. 5B. In some implementations, camera 5051 and the plurality of lightings 5055 are arranged so that a distance L2 between the position where the light paths of the plurality of lightings 5055 intersect and the FOV of camera 5051 are offset to camera 5051 by a distance of equal to or greater than 30 mm, for example, 30 mm, 33 mm, 35 mm, 37.5 mm, 40 mm, 42.4 mm, 45 mm, 48.8 mm, 50 mm, etc. This arrangement may enhance illumination of the camera and minimize dark shadows, spots, or edges.

In some implementations, camera module 505 may include a plurality of lightings spaced apart and surrounding the camera, and a lens hood surrounding the camera and positioned between the camera and the plurality of lightings. The spacing between adjacent lightings may or may not be the same. For example, when four lightings are provided, the angular spacing between lightings one and two and that between lightings two and three are 90 degrees apart, while that between lightings three and four is 120 degrees and that between lightings four and one is 60 degrees.

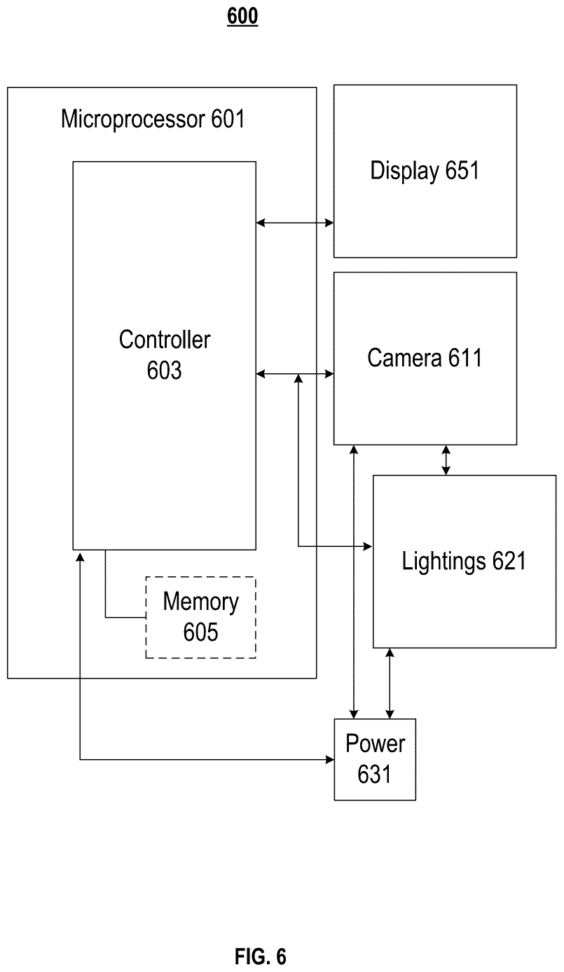

A system 600 of operating an oral imaging device is disclosed, as shown in FIG. 6. The oral imaging device can be any one of the oral imaging devices disclosed herein (e.g., oral imaging device 100, 300, 500, or the like). System 600 may include a microprocessor 601 coupled within the body (e.g., body 101, 301, or the like), a camera 611 (e.g., any camera disclosed herein) coupled to the microprocessor 601, one or more lightings 621 provided along with the camera module (e.g., any camera modules disclosed herein), and a power source 631 coupled to microprocessor 601, camera 611, and lightings 621. In some implementations, microprocessor 601 further include a controller 603 that is used to operate camera 611 and the plurality of lightings 621. Power source 631 may provide electric power to controller 603, camera 611, or lightings 621. In some implementations, power source 631 may be within the body (i.e., an internal power source) or may be connected to providing power outside the body (i.e., an external power source). That is, the power source may provide electric power to the oral imaging device via a wired or wireless connection.

In some implementations, microprocessor 601 may further include a memory 605 coupled to the body. Memory 605 may store the images captured by camera 611 or instructions or commands transmitted to and from controller 603. For example, memory 605 may store the combination of lightings being used during the examination, and controller 603 may control lightings 621 to turn on the corresponding light according to the instructions or commands input by a preset rule or by user selecting one or more different modes.

The foregoing description of the specific implementations reveals the general nature of the present disclosure that others can, by applying knowledge within the skill of the art, readily modify and/or adapt for various applications of such specific implementations, without undue experimentation, and without departing from the general concept of the present disclosure. Therefore, such adaptations and modifications are intended to be within the meaning and range of equivalents of the disclosed implementations, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Implementations of the present disclosure have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The Summary and Abstract sections may set forth one or more but not all exemplary implementations of the present disclosure as contemplated by the inventor(s), and thus, are not intended to limit the present disclosure and the appended claims in any way.

The breadth and scope of the present disclosure should not be limited by any of the above-described exemplary implementations, but should be defined only in accordance with the following claims and their equivalents.

What is claimed is:

1. An oral imaging device, comprising:
a body;
a support arm coupled to the body, wherein the support arm comprises an intra-oral end and a first guide structure; and
a camera module positioned at the intra-oral end of the support arm and configured to capture at least a first image including at least a first portion of a first dental arch,
wherein the support arm is stabilized by the first guide structure when the oral imaging device rests on a second dental arch.

2. The oral imaging device of claim 1, wherein the camera module is configured to move along a first trajectory.

3. The oral imaging device of claim 1, wherein the support arm further comprises a second guide structure,
wherein the camera module is configured to capture at least a second image including at least a second portion of the first dental arch or at least a portion of the second dental arch, and
wherein the support arm is stabilized by the second guide structure when the oral imaging device rests on the first dental arch or the second dental arch.

4. The oral imaging device of claim 3, wherein the camera module is configured to move along a second trajectory, and a plane of the second trajectory being different from that of the first trajectory.

5. The oral imaging device of claim 4, wherein the plane of the second trajectory and the plane of the first trajectory are perpendicular to each other.

6. The oral imaging device of claim 1, wherein the support arm further comprises a third guide structure, wherein the support arm is stabilized by the third guide structure when the oral imaging device rests on the same side as the first dental arch or the second dental arch, in order to capture at least a portion of the first dental arch or a portion of the second dental arch.

7. The oral imaging device of claim 6, wherein the third guide structure is on a first surface facing a first direction to which an aperture of a camera of the camera module is facing.

8. The oral imaging device of claim 1, wherein the first guide structure is on a second surface facing a second direction opposite to a first direction to which the aperture of the camera of the camera module is facing.

9. The oral imaging device of claim 1, wherein the oral imaging device is configured to rest on an anterior tooth of the first dental arch via the first guide structure to capture an occlusal surface of the second dental arch.

10. The oral imaging device of claim 1, wherein the first guide structure is configured to control the depth at which the camera module is inserted into the oral cavity or maintain a distance between the camera and the first dental arch to be captured.

11. The oral imaging device of claim 1, wherein the first guide structure comprises least one of a concave, a convex, or a bending structure.

12. The oral imaging device of claim 3, wherein the support arm has a first surface, second surface, and a third surface, the second surface is opposite to the first surface, and the third surface connects the first surface and the second surface,
wherein the camera module is on the first surface, and the second guide structure is on the third surface, and
wherein the third surface and the first surface are perpendicular to each other, and the first surface and the second surface are parallel to each other.

13. The oral imaging device of claim 3, wherein the second guide structure comprises a plurality of concaves, or a plurality of convexes, or a combination thereof, configured to accommodate dental arches of different sizes.

14. The oral imaging device of claim 1, wherein a surface of the first guide structure contacting the dental arch is smooth in a width direction.

15. The oral imaging device of claim 1, wherein the camera module comprises:
a camera, comprising a wide-angle camera.

16. The oral imaging device of claim 15, wherein a field of view (FOV) of the camera is equal to or larger than 140°.

17. The oral imaging device of claim 15, wherein the camera has a rectangular FOV, and a shorter side of the rectangular FOV is parallel to a length direction of the support arm.

18. The oral imaging device of claim 1, wherein an angle between the optical axis of a camera of the camera module and a length direction of the body is between 40° to 90°.

19. The oral imaging device of claim 1, wherein the camera module comprises a camera and a plurality of lightings spaced apart and surrounding the camera,
wherein the camera and the plurality of lightings are arranged so that a distance between the camera and an intersection of light paths of the plurality of lightings is equal to or less than 10 mm, and
wherein the camera and the plurality of lightings are arranged so that a distance between the position where the light paths of the plurality of lightings and the FOV of the camera are offset to the camera is equal to or greater than 30 mm.

20. The oral imaging device of claim 1, further comprising:
a gag, wherein an opening of the gag is configured to correspond to an aperture of a camera of the camera module to allow the camera to capture the image via the opening of the gag, wherein a relative position between the gag and the support arm is adjustable.
